(19) United States
(12) Patent Application Publication
(10) Pub. No.: US 2014/0371562 A1
(43) Pub. Date: Dec. 18, 2014

(54) SOLDERABLE FLEXIBLE ELECTRODE AND METHOD FOR MANUFACTURING SAME

(71) Applicant: Korea University Research and Business Foundation, Seoul (KR)

(72) Inventors: Sang Hoon Lee, Gyeonggi-do (KR); Gi-Seok Jeong, Gyeongsangbuk-do (KR); Dong Hyun Baek, Chungcheongnam-do (KR); Hachul Jung, Seoul (KR)

(73) Assignee: Korea University Research and Business Foundation, Seoul (KR)

(21) Appl. No.: 14/356,092

(22) PCT Filed: Sep. 28, 2012
(86) PCT No.: PCT/KR2012/007908

§ 371 Date: May 2, 2014

(30) Foreign Application Priority Data
Nov. 3, 2011 (KR) 10-2011-0114069

(51) Int. Cl. A61B 5/00; B05D 7/00

(52) U.S. Cl. CPC A61B 5/6801; B05D 7/5883
USPC 600/372; 427/2.12

(57) ABSTRACT

The present disclosure relates to a flexible electrode and a method for manufacturing the same, more particularly to a flexible electrode which has good flexibility and biocompatibility as well as good solderability for attachment of electronic parts and, hence, is widely applicable in such fields as ubiquitous healthcare monitoring and a method for manufacturing the same.

### TECHNICAL FIELD

The present disclosure relates to a flexible electrode and a method for manufacturing the same, more particularly to a flexible electrode which has good flexibility and biocompatibility as well as good solderability for attachment of electronic parts and, hence, is widely applicable in such fields as ubiquitous healthcare monitoring and electric circuitry for flexible displays and a method for manufacturing the same.

### BACKGROUND ART

With the recent development in communications technology, industries related to ubiquitous healthcare monitoring which allows easy monitoring and diagnosis of a subject anytime, anywhere are growing remarkably. In addition, the human-machine communications, or human body communications, technology is attracting a lot of interests as a core technology in the development of wearable computers, wearable displays or devices for smart surgery such as tactile sensors.

For an electrode to be used in such applications, it should have superior ductility, malleability, bendability or foldability so as to keep contact with human skin at all times or on occasions and should not be greatly affected by the change in electrical resistance due to excretion from the human body, such as sweat. In addition, the electrode should not cause adverse reactions such as inflammation, necrosis, etc. of the skin even after long-time wearing.

Although the currently developed or commercialized flexible electrodes have bendability or other properties to some extent, they fail to satisfy all of the above-described characteristics and are limited in actual application in related industries. To overcome this limitation, researches are actively under way on an electrode having further improved flexibility and circuitry using the same. At present, a method of preparing polydimethylsiloxane (PDMS) into an electrode of a particular shape, a method of preparing an electrode by depositing metal ions on the surface of PDMS, a method of preparing an electrode by mixing a metal with a polymer, or a method of preparing an electrode by mixing a carbon nanotube with a polymer, etc. have been developed. And, recently, a method of preparing an electrode using graphene was been reported. In this regard, Korean Patent Publication No. 2011-0108194 discloses a flexible current collector for an electrode, a method for preparing the same and a negative electrode using the same, wherein the current collector for an electrode includes a flexible polymer substrate, a crosslinkable polymer layer and a metal layer, the surface of the polymer layer having a plurality of heights and grooves formed thereon. Specifically, it is described that the plurality of heights and grooves can be formed using PDMS having a corresponding shape.

Although the currently developed technologies provide improved flexibility to some extent, there are still limitations in attaching various electronic parts. That is to say, most of the currently used electronic parts which are attached to circuitry through soldering cannot be connected to a flexible electrode. It is because, although an electrode made of a metallic material can be attached on a flexible surface such as PDMS, the bonding strength is not so high. Thus, if the flexible electrode is bent or stretched after soldering, the electrode is easily detached from the PDMS surface. Accordingly, there is a need of the development of an electrode being solderable and thus exhibiting superior connectivity to electronic parts, while exhibiting superior flexibility and biocompatibility.

### DISCLOSURE

### Technical Problem

The present disclosure is directed to providing a flexible electrode exhibiting superior solderability such that electronic parts can be attached thereto, while exhibiting superior flexibility and biocompatibility.

The present disclosure is also directed to providing a method for manufacturing the flexible electrode.

### Technical Solution

In a general aspect the present disclosure provides a flexible electrode including:
a porous polymer support layer;
   a first metal deposition layer formed on the porous polymer support layer;
a second metal deposition layer formed on the first metal deposition layer; and
a metal plating layer formed on the second metal deposition layer.

In an exemplary embodiment of the present disclosure, a polymer forming the porous polymer support layer may be silicone rubber, a metal forming the first metal deposition layer may be titanium, chromium or an alloy thereof, a metal forming the second metal deposition layer may be selected from a group consisting of gold, silver and copper, and a metal forming the metal plating layer may be selected from a group consisting of nickel, gold, silver and copper.

In another exemplary embodiment of the present disclosure, the polymer support layer may have a thickness of 100-200 μm, the first metal deposition layer may have a thickness of 200-500 Å, and the second metal deposition layer may have a thickness of 100-5000 Å.

In another exemplary embodiment of the present disclosure, the porous polymer may have pores of 1-100 μm in diameter and depth.

In another exemplary embodiment of the present disclosure, the present disclosure may further include a conductivity-improving and oxidation-preventing layer on the metal plating layer.

In another general aspect, the present disclosure provides a method for manufacturing a flexible electrode, including:
forming a polymer support layer on a substrate;
forming a porous polymer support layer from the polymer support layer;
forming a first metal deposition layer on the porous polymer support layer;
forming a second metal deposition layer on the first metal deposition layer; and
forming a metal plating layer on the second metal deposition layer.

In an exemplary embodiment of the present disclosure, the polymer support layer may be formed on the substrate by spin coating.

In another exemplary embodiment of the present disclosure, the porous polymer support layer may be formed by injecting steam to a polymer.

In another exemplary embodiment of the present disclosure, a polymer forming the porous polymer support layer may be silicone rubber, a metal forming the first metal deposition layer may be titanium, chromium or an alloy thereof, a metal forming the second metal deposition layer may be selected from a group consisting of gold, silver and copper, and a metal forming the metal plating layer may be selected from a group consisting of nickel, gold, silver and copper.

In another exemplary embodiment of the present disclosure, the method for manufacturing a flexible electrode according to the present disclosure may further include forming a conductivity-improving and oxidation-preventing layer on the metal plating layer.

In another exemplary embodiment of the present disclosure, the porous polymer support layer may be formed according to a pattern to form a patterned porous polymer support layer.

In another exemplary embodiment of the present disclosure, the first metal deposition layer, the second metal deposition layer or the metal plating layer may be formed according to a pattern to form a patterned first metal deposition layer, a patterned second metal deposition layer or a patterned metal plating layer.

### Advantageous Effects

in accordance with the present disclosure, a flexible electrode exhibiting superior solderability such that electronic parts can be attached thereto, while exhibiting superior flexibility and biocompatibility, and a method for manufacturing the same may be provided.

### BRIEF DESCRIPTION OF DRAWINGS

FIGS. 1a-1e schematically show by what mechanism a metal plating layer is mechanically bonded to a porous polymer support layer during formation of the metal plating layer.

FIGS. 2a-2c are electron micrographs of a flexible electrode according to the present disclosure.

FIGS. 3a-3f schematically show a procedure from formation of a polymer support layer to formation of a second metal deposition layer in a manufacturing method according to the present disclosure.

FIGS. 4a-4d schematically show a procedure of forming a porous polymer support layer having a predetermined pattern (FIG. 4a), forming first and second metal deposition layers thereon (FIG. 4b), completing a flexible electrode according to the present disclosure by forming a metal plating layer (FIG. 4c), and connecting LED electronic parts to the completed flexible electrode by soldering (FIG. 4d).

FIGS. 5a-5b show a result of stretching (FIG. 5a) and bending (FIG. 5b) tests of a display device prepared by the method shown in FIGS. 4a-4d.

FIG. 6 shows an image of an electrode including a porous PDMS support layer, a black titanium layer and a gold layer attached to a human finger.

FIG. 7 shows a result of bending and stretching a human finger several times with a flexible electrode according to the present disclosure attached thereto.

FIGS. 8a-8d show a flexible electrode according to the present disclosure worn by a subject for use (FIG. 8a), the state of the skin of a subject after wearing the electrode for a week (FIG. 8b), ECG of a subject measured using an electrode according to the present disclosure during rest (FIG. 8c), and ECG measured using a commercially available electrode (Ag/AgCl) (FIG. 8d).

FIGS. 9a-8b show ECGs measured during rest, during walking and during perspiration after exercise using an electrode according to the present disclosure (FIG. 9a) or a commercially available electrode (Ag/AgCl) (FIG. 9b).

FIG. 10 shows ECGs measured on days 1, 3, 5 and 7 while a subject wears a flexible electrode according to the present disclosure for a week.

### BEST MODE FOR CARRYING OUT INVENTION

Hereinafter, the present disclosure is described in more detail.

A flexible electrode according to the present disclosure includes: a porous polymer support layer; a first metal deposition layer formed on the porous polymer support layer; a second metal deposition layer formed on the first metal deposition layer; and a metal plating layer formed on the second metal deposition layer.

In the flexible electrode according to the present disclosure, the porous polymer support layer serves to provide flexibility to the flexible electrode. Accordingly, any polymer material capable of ensuring superior flexibility may be used in the porous polymer support layer. For example, most silicone rubber such as polydimethylsiloxane (PDMS) may be used. The porous polymer support layer may be formed to any thickness. Specifically, it may be formed to a thickness of 10 μm to 1 mm, so that suitable flexibility can be conferred while maintaining the mechanical strength of the flexible electrode. If the thickness of the porous polymer support layer is smaller than 10 μm, pores may not be formed on the polymer surface as desired and, as a result, mechanical strength required for the flexible electrode may not be achieved. But, when a very flexible electrode requiring no resistance to large external force is to be prepared, the porous polymer support layer may be formed to a thickness smaller than 10 μm. And, if the thickness of the porous polymer support layer exceeds 1 mm, flexibility decreases. But, when an electrode requiring high mechanical strength is to be prepared, the porous polymer support layer may be formed to a thickness greater than 1 mm.

In the flexible electrode according to the present disclosure, metals used to form the first metal deposition layer and the second metal deposition layer may be conducting metals commonly used to prepare a flexible substrate. For example, a metal forming the first metal deposition layer may be titanium, chromium or an alloy thereof, and a metal forming the second metal deposition layer may be selected from a group consisting of gold, silver and copper, although not being limited thereto. The first metal deposition layer serves to prevent the conducting metal layers from being separated from the polymer support layer even when the electrode is bent or folded by improving bonding strength between the second metal deposition layer and the porous polymer support layer. The second metal deposition layer serves to Improve the conductivity of the flexible electrode.

The first metal deposition layer may have a thickness of 200-800 Å. If the thickness is smaller than 200 Å, bonding strength may decrease because the mechanical strength of the metal deposition layer is too low. And, if it exceeds 500 Å, flexibility may decrease. The second metal deposition layer may have a thickness of 100-5000 Å. If the thickness is smaller than 100 Å, electrical resistance may be too high. And, if it exceeds 5000 Å, flexibility may decrease. However, the thicknesses of the first and second metal deposition layers may be outside the aforedescribed ranges, depending on the use, required flexibility, etc. of the electrode.

The flexible electrode according to the present disclosure includes a metal plating layer formed on the second metal deposition layer. The metal plating layer is located between the pores of the porous polymer support layer and, hence, the metal plating layer is mechanically bonded to the porous polymer support layer. Owing to the mechanical bonding, the flexible electrode according to the present disclosure has superior solderability.

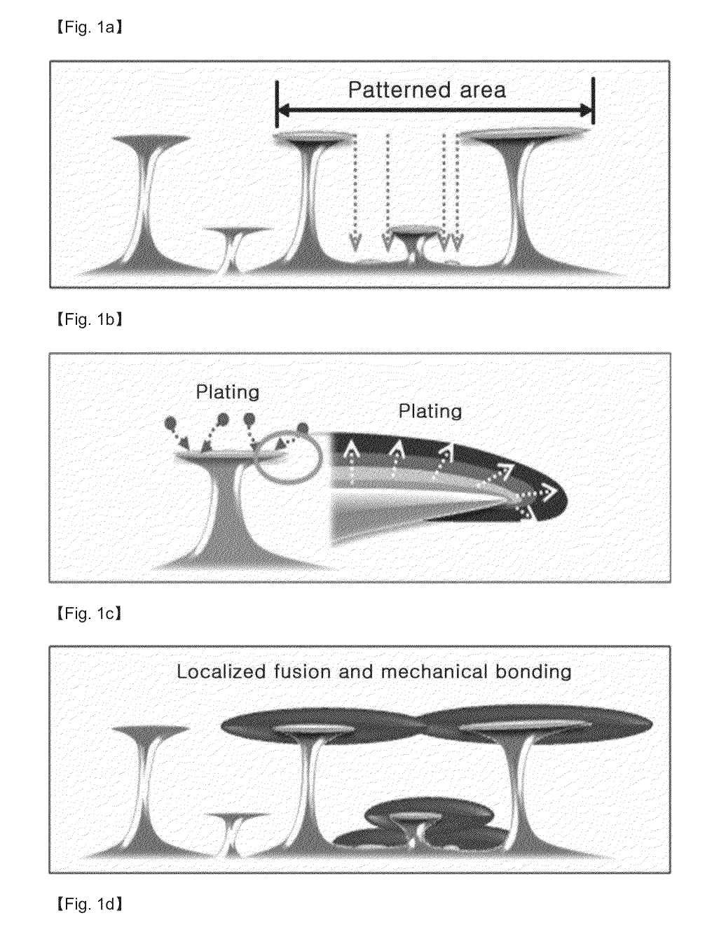

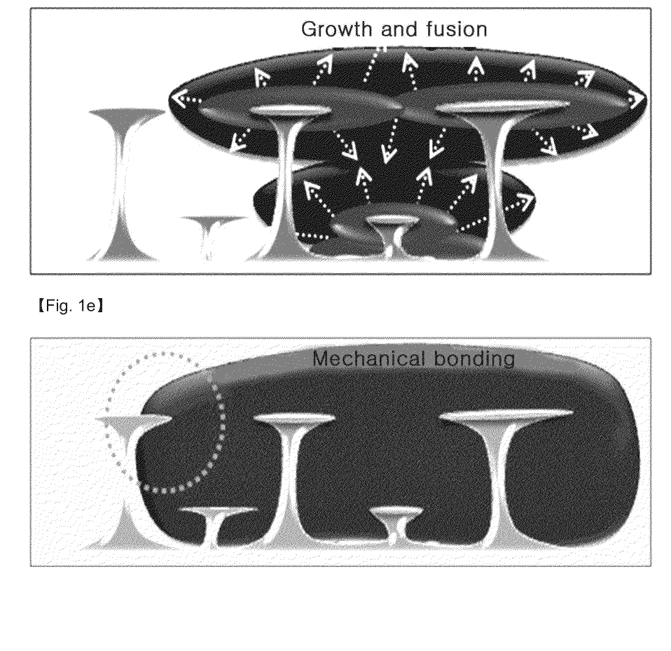

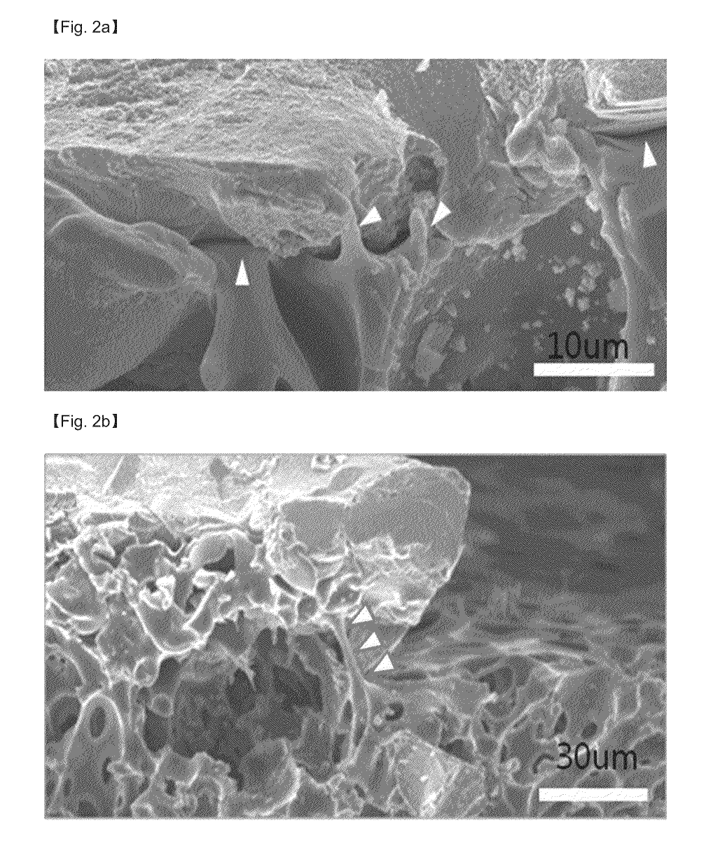

FIGS. 1a-1e schematically show by what mechanism the metal plating layer is mechanically bonded to the porous polymer support layer during formation of the metal plating layer. Referring to the figures, plating is conducted after the first and second metal deposition layers have been formed on the patterned porous polymer support layer. In the early stage of plating, plating is achieved partially only on the patterned upper portion of the porous polymer on which the first and second metal deposition layers have been formed and localized fusion and mechanical bonding occur at the upper portion (see FIG. 1c). As the plating process advances, the patterned upper portion and the patterned lower portion on the porous surface grow and bond with each other. As a result, a metal layer is formed in both the upper and lower portions of the pores and fills the pores on the surface. As a result, the metal layer is mechanically bonded to the surface of the porous polymer (see FIG. 1d). After the formation of the metal plating layer is completed, mechanical bonding with other adjacent patterns is enhanced since the patterns of the porous polymer support layer are located between the patterns of the metal plating layer. And, as shown in the figures, since the pattern of the porous polymer support layer has a ring shape and fixes the metal plating layer to the porous polymer support layer so that they are not easily separated, mechanical bonding between the metal plating layer and the porous polymer support layer is also improved significantly (see FIG. 1e). The improvement in mechanical bonding can be visually observed from the electron micrographs shown in FIGS. 2a-2c.

A metal forming the metal plating layer may be any metal as long as it has superior palatability and mechanical strength suitable for soldering and can extend between the pores of the polymer support layer. For example, it may be a metal selected from a group consisting of nickel, gold, sliver and copper, although not being limited thereto.

If necessary, the flexible electrode according to the present disclosure may further include a conductivity-improving and oxidation-preventing layer formed on the metal plating layer in order to improve the conductivity of the electrode and prevent oxidation. The conductivity-improving and oxidation-preventing layer serves to reduce the contact resistance with the metal plating layer which has grown from plating and prevent oxidation of the metal plating layer. The conductivity-improving and oxidation-preventing layer may be formed from a metal that can confer oxidation resistance and is easily solderable, such as silver or gold, although not being limited thereto.

In another aspect, the present disclosure provides a method for manufacturing a flexible electrode, including: forming a polymer support layer on a substrate; forming a porous polymer support layer from the polymer support layer; forming a first metal deposition layer on the porous polymer support layer; forming a second metal deposition layer on the first metal deposition layer; and forming a metal plating layer on the second metal deposition layer.

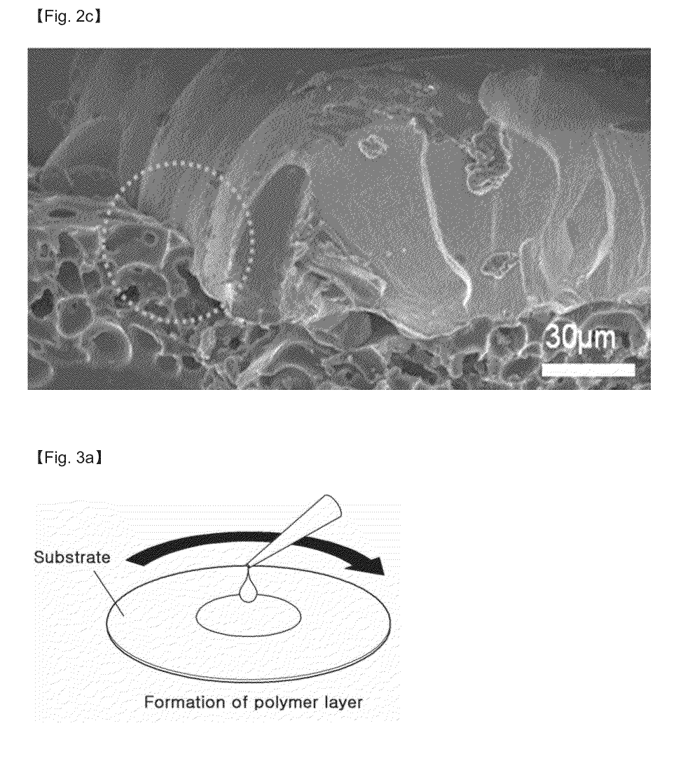

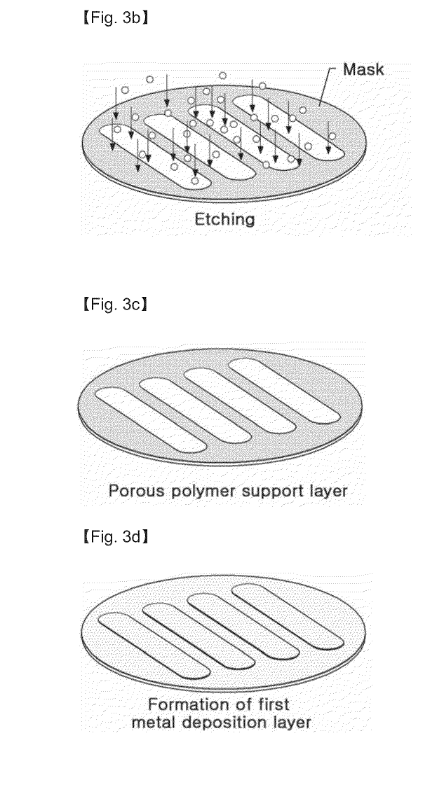

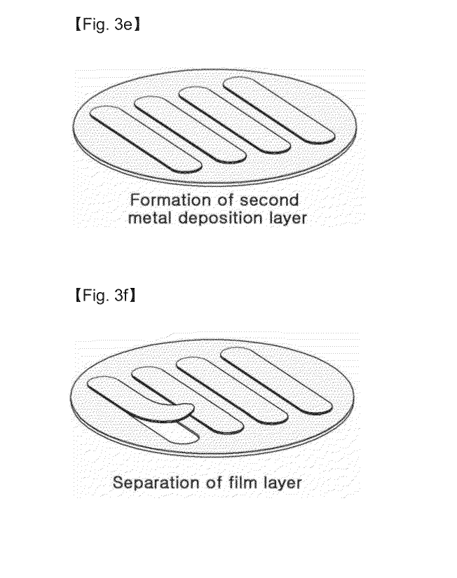

FIGS. 3a-3f schematically show the procedure from formation of the polymer support layer to formation of the second metal deposition layer in the manufacturing method according to the present disclosure. The method for manufacturing a flexible electrode according to the present disclosure will be described in detail referring to the figures. The constituent material and thickness of each layer are the same as described above.

Referring to FIG. 3a, a material forming the polymer support layer is coated on a commonly used substrate, such as a silicon wafer, glass, etc. The polymer support layer may ho formed by a commonly employed method of coating a polymer on a substrate, although not being limited thereto. For example, spin coating or other methods may be used. The polymer may be coated by spin coating such that the porous polymer support layer has a thickness of 10 μm-1 mm in the finally completed flexible electrode. As described above, the porous polymer support layer may have a thickness of not greater than 1 mm to ensure the flexibility of the electrode. But, when an electrode requiring high mechanical strength is to be prepared, the porous polymer support layer may be formed to a thickness greater than 1 mm.

Referring to FIG. 3b, after the polymer support layer has been formed on the substrate, a mask having a desired predetermined pattern is placed on the polymer support layer and a porous surface is formed. Although FIG. 3b shows an embodiment wherein the porous polymer support layer is formed only on the predetermined pattern, the present disclosure is not limited thereto. Differently from FIG. 3b, the porous polymer support layer may be formed on the entire surface of the polymer support layer and then a pattern may be formed in the following process of metal layer deposition or plating.

Specifically, the porous polymer layer may be formed by injecting steam to a thermosetting polymer. For example, it may be achieved by injecting steam of 100-150° C. and 60-100 kPa for 1-10 minutes. However, the temperature and pressure of the steam and the injection time can vary depending on the thermal deformation characteristics of the polymer. The preparation of the porous polymer layer using steam can be performed very easily at low cost and the formed porous polymer layer has a mushroom-shaped cross section as shown in FIG. 1a and thus provides very strong mechanical bonding between the metal layer and the polymer layer.

After the preparation of the porous polymer support layer has been completed by forming the porous surface (see FIG. 3c), the first metal deposition layer (see FIG. 3d) and the second metal deposition layer (see FIG. 3e) are deposited sequentially. The first metal deposition layer and the second metal deposition layer are formed by deposition because it is easy to finely pattern the metal deposition layers on the polymer surface. After the deposition of the porous polymer support layer, the first metal deposition layer and the second metal deposition layer, plating is conducted after the electrode is separated from the substrate (or before it is separated from the substrate). As described above, if necessary, the conductivity-improving and oxidation-preventing layer for improving the conductivity of the electrode and preventing oxidation may be further formed.

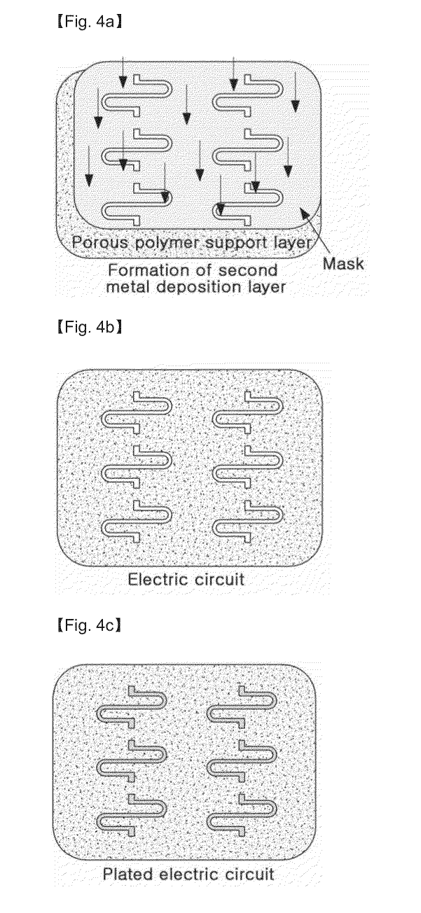

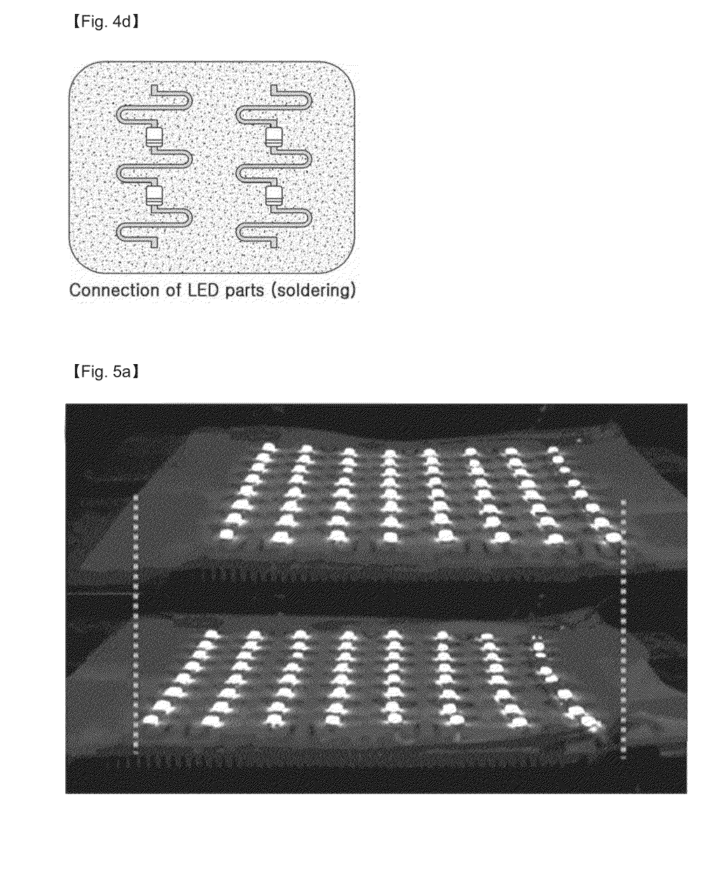

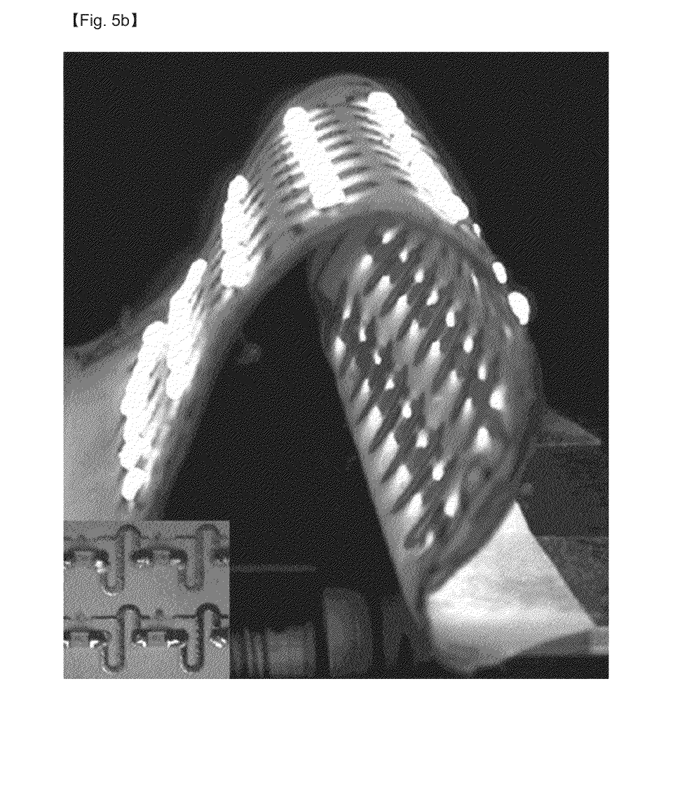

In the present disclosure, the metal plating layer for improving solderability is formed on the second metal deposition layer in order to make connection of electronic parts easier. The metal plating layer may be formed by a commonly employed plating method such as electroplating. FIGS. 4a-4d schematically show a procedure of forming the porous polymer support layer having a predetermined pattern (FIG. 4a), forming the first and second metal deposition layers thereon (FIG. 4b), completing the flexible electrode according to the present disclosure by forming the metal plating layer (FIG. 4c), and connecting LED electronic parts to the completed flexible electrode by soldering (FIG. 4d). And, FIGS. 5a-5b show a result of stretching (FIG. 5a) and bending (FIG. 5b) tests of a display device prepared by the method shown in FIGS. 4a-4d. As can be seen from FIGS. 5a-5b, the flexible electrode according to the present disclosure allows connection of electronic parts through soldering while having superior flexibility.

### MODE FOR CARRYING OUT INVENTION

Hereinafter, the present disclosure will be described in detail through an example. However, the following example is for illustrative purposes only and it will be apparent to those of ordinary skill in the art that the scope of the present disclosure is not limited by the example.

### EXAMPLE

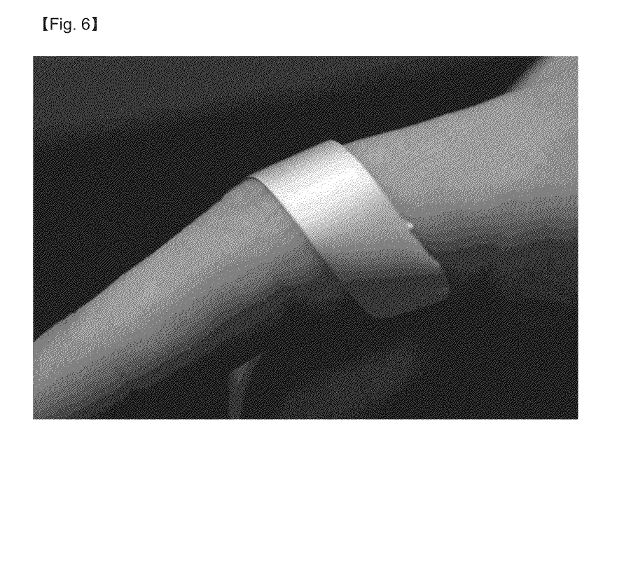

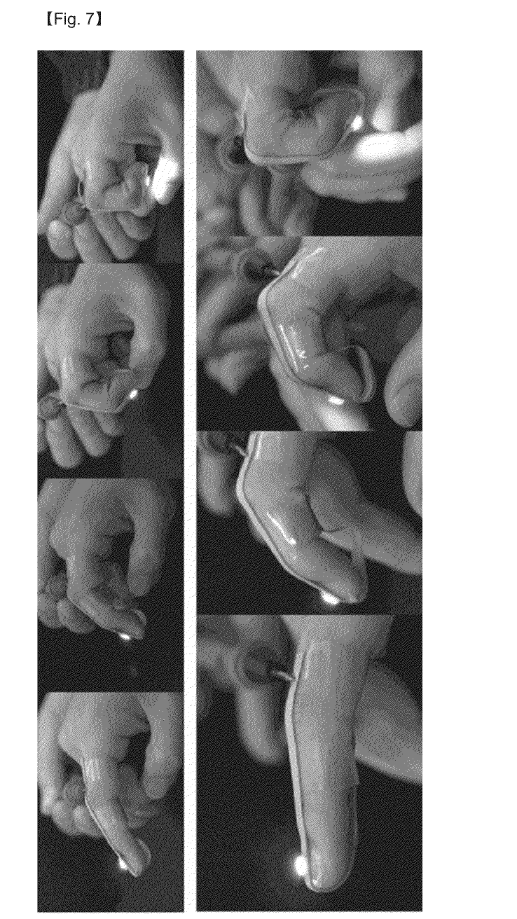

A 100-μm thick master mold was prepared by spin coating SU-8 (50) on a silicon substrate at 1000 rpm. Then, polydimethylsiloxane was injected into the master mold to a desired thickness. Subsequently, steam was injected using a commonly used pressure cooker (110-120° C., 70-50 kPa) for 1 minute, with the spacing with the polydimethylsiloxane maintained within 1 cm. After the steam treatment, a porous polymer support was prepared by drying in an oven at 80° C. for 1 hour to remove moisture. On the patterned porous PDMS layer, a first metal deposition layer of titanium (iTASCO) (thickness: 200 Å, deposition speed: 0.5 Å/s) and a second metal deposition layer of gold (Taein, 99.99%, thickness: 3000 Å, deposition speed: 1 Å/s) were deposited using electron beams (SNT, pressure: 6×10⁻⁸ torr). FIG. 6 shows an image of the prepared electrode attached to a human finger. As can be seen from FIG. 6, the electrode having the porous PDMS support layer, the black titanium layer and the gold layer formed could be attached to the finger with superior flexibility.

Subsequently, the patterned area with the black titanium layer and the gold layer formed on the porous PDMS support layer was separated from the silicon wafer and a Ni plating layer was formed by electroplating (electroplating apparatus: Keithly 2400, nickel plating solution: 300 g/L nickel sulfate, 57 g/L nickel chloride, 57 g/L boric acid, plating bath temperature: 50° C., current density: 2 A/dcm²).

FIG. 7 shows a result of bending and stretching a human finger several times with the prepared flexible electrode attached thereto. As can be seen from FIG. 7, the flexible electrode according to the present disclosure having the porous PDMS support layer, the black titanium layer, the gold layer and the nickel layer formed could be attached to the finger with superior flexibility and exhibited superior electrical conductivity when LED parts were connected through soldering.

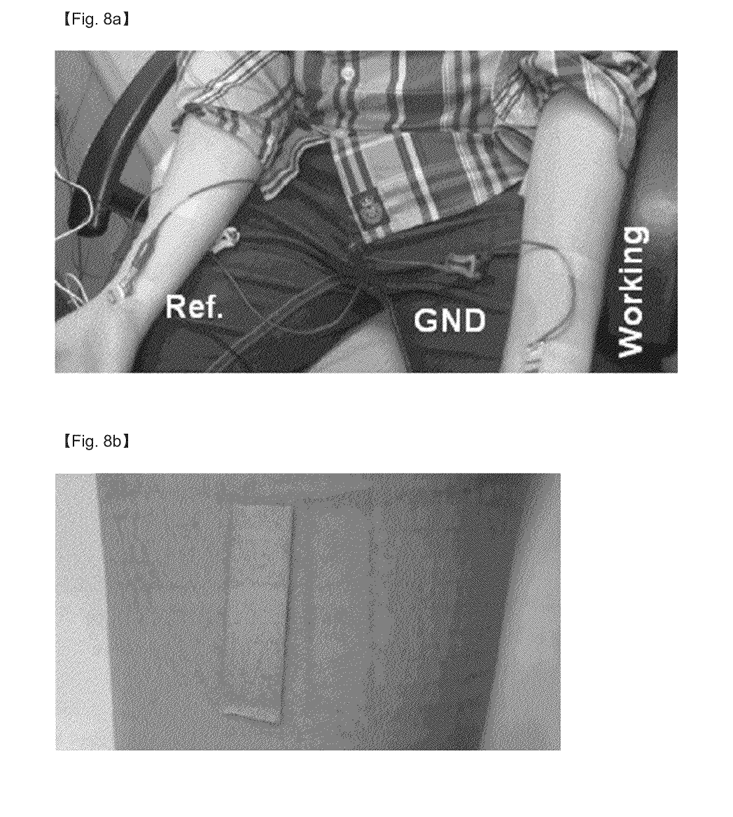

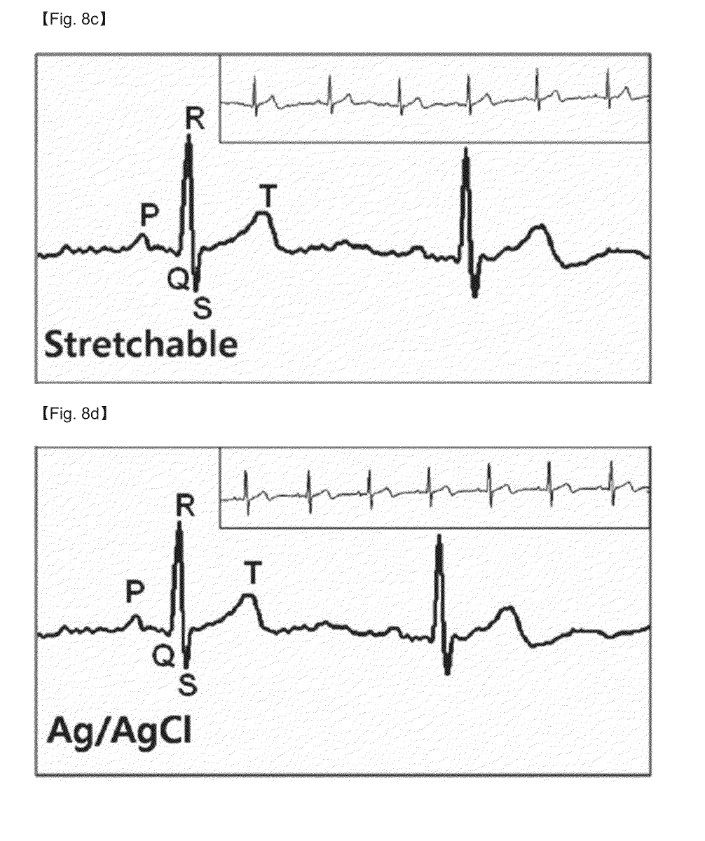

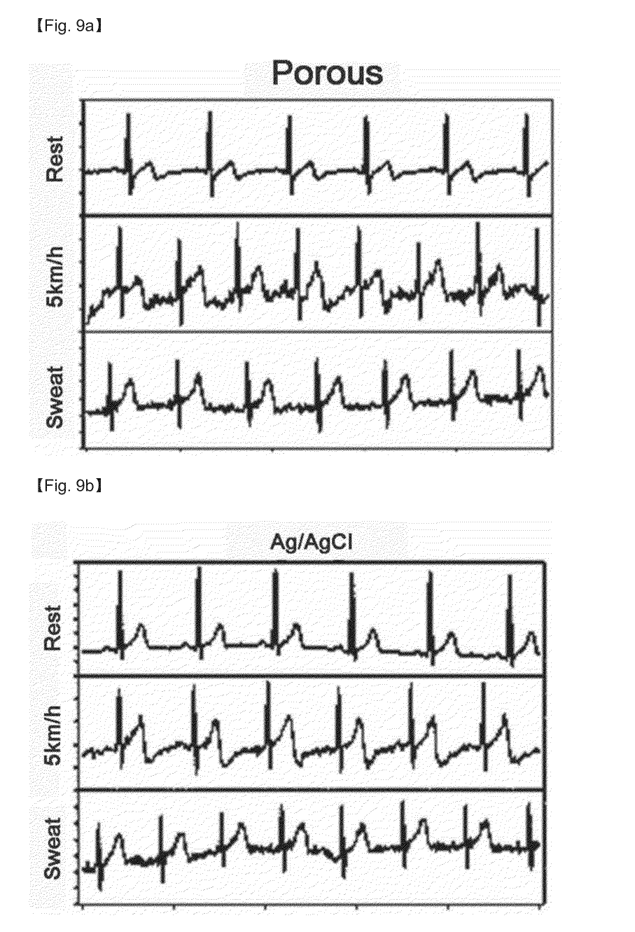

Electrocardiogram (ECG) was measuring using the electrode of the present disclosure. The ECG measurement was made with a reference electrode connected to the right arm, a working connected to the left arm and a ground electrode connected to the left foot. The reference electrode and the working electrode were the electrode formed on the porous PDMS layer and the ground electrode was a commercially available Ag/AgCl electrode. FIGS. 8a-8d show the flexible electrode according to the present disclosure worn by a subject for use (FIG. 8a), the state of the skin of the subject after wearing the electrode for a week (FIG. 8b), ECG of the subject measured using the electrode according to the present disclosure during rest (FIG. 8c), and ECG measured using the commercially available electrode (Ag/AgCl) (FIG. 8d). As can be seen from the figures, the electrode according to the present disclosure did not induce skin troubles such as red spots even after long-term use (see FIG. 8b) and could measure ECG with superior performance when compared with the commercially available electrode (see FIGS. 8c-8d).

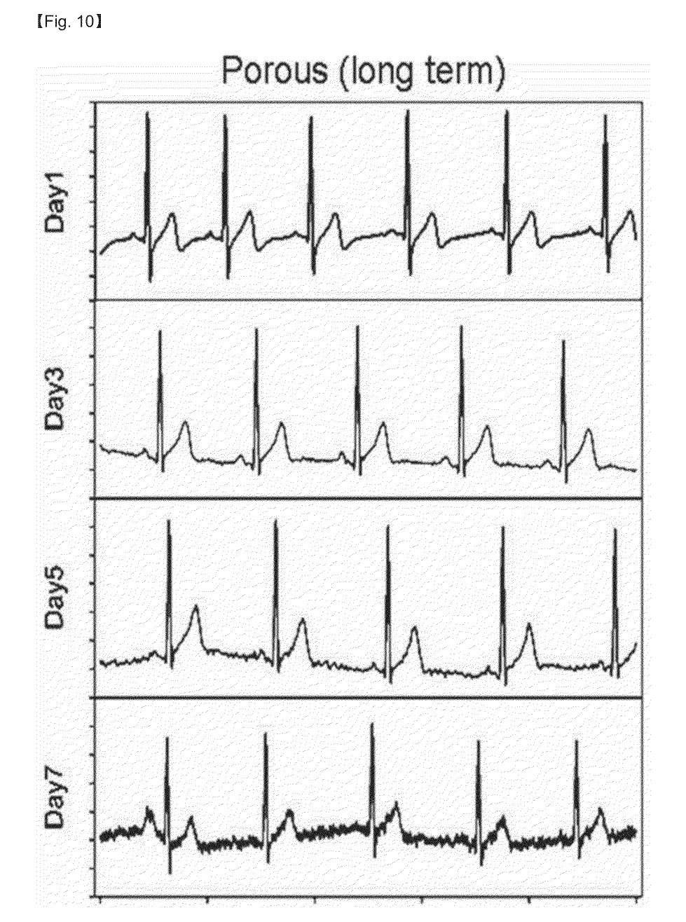

FIGS. 9a-9b show ECGs measured during rest, during walking and during perspiration after exercise using the electrode according to the present disclosure (FIG. 9a) or the commercially available electrode (Ag/AgCl) (FIG. 9b). It can be seen from the figures that ECG could be measured with superior performance using the electrode according to the present disclosure. FIG. 10 shows ECGs measured on days 1, 3, 5 and 7 while a subject wears the flexible electrode according to the present disclosure for a week. It can be seen from the figure that the flexible electrode according to the present disclosure provides stable ECG data even when worn for a long period of time.

### INDUSTRIAL APPLICABILITY

A flexible electrode according to the present disclosure allows connection of commonly used electronic parts through, for example, soldering, while having superior flexibility.

1. A flexible electrode comprising:
a porous polymer support layer:
a first metal deposition layer formed on the porous polymer support layer;
a second metal deposition layer formed on the first metal deposition layer; and
a metal plating layer formed on the second metal deposition layer.

2. The flexible electrode according to claim 1, wherein a polymer forming the porous polymer support layer is silicone rubber, a metal forming the first metal deposition layer is titanium, chromium or an alloy thereof, a metal forming the second metal deposition layer is selected from a group consisting of gold, silver and copper, and a metal forming the metal plating layer is selected from a group consisting of nickel, gold, sliver and copper.

3. The flexible electrode according to claim 1, wherein the polymer support layer has a thickness of 100-200 μm, the first metal deposition layer has a thickness of 200-500 Å, and the second metal deposition layer has a thickness of 100-5000 Å.

4. The flexible electrode according to claim 1, wherein the porous polymer has pores of 1-100 μm in diameter and depth.

5. The flexible electrode according to claim 1, which further comprises a conductivity-improving and oxidation-preventing layer on the metal plating layer.

6. A method for manufacturing a flexible electrode, comprising:
forming a polymer support layer on a substrate;
forming a porous polymer support layer from the polymer support layer;
forming a first metal deposition layer on the porous polymer support layer;
forming a second metal deposition layer on the first metal deposition layer; and
forming a metal plating layer on the second metal deposition layer.

7. The method for manufacturing a flexible electrode according to claim 6, wherein the polymer support layer is formed on the substrate by spin coating.

8. The method for manufacturing a flexible electrode according to claim 6, wherein the porous polymer support layer is formed by injecting steam to a polymer.

9. The method for manufacturing a flexible electrode according to claim 6, wherein a polymer forming the porous polymer support layer is silicone rubber, a metal forming the first metal deposition layer is titanium, chromium or an alloy thereof, a metal forming the second metal deposition layer is selected from a group consisting of gold, silver and copper, and a metal forming the metal plating layer is selected from a group consisting of nickel, gold, silver and copper.

10. The method for manufacturing a flexible electrode according to claim 6, which further comprises forming a conductivity-improving and oxidation-preventing layer on the metal plating layer.

11. The method for manufacturing a flexible electrode according to claim 6, wherein the porous polymer support layer is formed according to a pattern to form a patterned porous polymer support layer.

12. The method for manufacturing a flexible electrode according to claim 6, wherein the first metal deposition layer, the second metal deposition layer or the metal plating layer is formed according to a pattern to form a patterned first metal deposition layer, a patterned second metal deposition layer or a patterned metal plating layer.
